(19) United States
(12) Patent Application Publication
(10) Pub. No.: US 2006/0079791 A1
(43) Pub. Date: Apr. 13, 2006

(54) Micro blood pressure sensor and measuring instrument using same

(75) Inventors: Roland Letremy, Thilouze (FR); Pierre Boutouyrie, Paris (FR); Dominique Lemoine, Tours (FR); Stephane Laurent, Paris (FR); Robert Corvisier, Ballan Mire (FR)

Correspondence Address: OBLON, SPIVAK, MCCLELLAND, MAIER & NEUSTADT, P.C., 1940 DUKE STREET, ALEXANDRIA, VA, 22314, US

(73) Assignee: INSTITUT NAT. DE LA SANTE ET DE LA RECHER. MED, Paris 15eme (FR)

(21) Appl. No.: 10/546,039

(22) PCT Filed: Feb. 23, 2004
(86) PCT No.: PCT/FR04/50073

§ 371 Date: Aug. 17, 2005

(30) Foreign Application Priority Data
Feb. 26, 2003 (FR) 0302342

(51) Int. Cl. A61B 5/02

(52) U.S. Cl. 600/485; 600/500

(57) ABSTRACT

The invention relates to a blood pressure microsensor comprising means of holding and positioning a chip of piezoresistive material (10) on the inside face of a practitioner's finger, the chip dimensions being smaller than the diameter of the artery in which the pressure is to be measured, the microsensor also comprising means of transmitting the electrical signal output (12) by the piezoresistive chip in response to a pressure applied to the chip (10). It also relates to an arterial stiffness measurement apparatus using such microsensors.

### TECHNICAL DOMAIN

The invention relates to a blood pressure microsensor that can be used to determine a cardiovascular risk factor. It also relates to an instrument for measuring the arterial stiffness using such microsensors.

### STATE OF PRIOR ART

Cardiovascular diseases are still the leading cause of death in developed countries, and are becoming the leading cause of death throughout the world.

At the moment, it is very difficult to predict the probability of occurrence of a cardiovascular disease for an individual over a time scale of ten or twenty years. Many cardiovascular risk factors have been identified during longitudinal monitoring studies, particularly in North America (Framingham Heart Study). The main risk factors are age, sex, hypercholesterolemy, high blood pressure, diabetes, and smoking. There are also many other biological risk factors (like Hyperhomocysteinemia, chronic inflammation: CRP, contents of heavy metals, etc.), and socio-economic risk factors (level of education, type of work, place of residence, etc.). However, the prediction is still very bad even when all of these identified risk factors are within a risk prediction score, particularly for an individual person. In other words, most cardiovascular events will occur in people wrongly considered to have a low cardiovascular risk. There are many explanations for this phenomenon. The most obvious among them is that the very concept of a risk factor is based on a statistical deviation in the general population, which by definition means that patients with large anomalies in the parameter in question (that represents a high risk factor) will be selected, but these patients only represent a tiny proportion of the total population. The other equally trivial limit is that biological risk factors are variable with time, thus acting as an integrator.

Therefore it is essential to promote new risk markers including all identified or non-identified risk factors during the life of an individual. Structure parameters and large artery function parameters (arteries affected by atheroma) are the most promising so-called “integrators”. The two factors about which there are most arguments at the present time are the intima media thickness of the primitive carotid artery, which we are not interested in herein, and the stiffness of large arteries. The large arteries close to the heart have the property of deforming during pressure changes related to the heart contraction. The proportionality ratio between the deformation and the deforming force corresponds to the arterial stiffness. The physiological role of the arterial elasticity (stiffness) is very important. The elasticity of large arteries acts as a diastolic relay to heart contraction. The heart only contracts during a third of a cycle (systole). The potential energy transferred to the blood during systole is transmitted to the wall of the large arteries in the form of an elastic deformation and the large arteries restore this energy during diastole, thus contributing to circulation of the blood. The stiffness of the arteries increases with age, and thus with most cardiovascular risk factors identified at the moment.

The pulse wave velocity (pressure wave transit time between two arterial points, conventionally the primitive carotid artery in the neck and the common femoral artery at the groin) has been known longer and has been better validated than any other arterial stiffness parameter. It was recently demonstrated that the pulse wave velocity predicted the occurrence of an ischemic heart disease and cardiovascular mortality, independently of and beyond the prediction made through conventional risk factors.

Adjacent to the pulse wave velocity, an analysis of the carotid pressure wave can provide interesting hemodynamic parameters. In particular, the reflection percent of the pulsatile wave can be quantified by measuring the amplification index. This parameter is currently being epidemiologically validated.

The velocity of the pulse wave was used by Bramwell and Hill as an arterial distensibility index in 1922, and since then by many other authors.

The relation between the pulse wave transmission velocity (that should be distinguished from the blood flow velocity) and the elastic properties of the arterial wall has been extensively studied theoretically and experimentally. The velocity of the pulse wave is proportional to the square root of the Young's modulus of the material from which the wall is made (Moens-Korteweg equation).

FIG. 1 illustrates the technique for measuring the carotid femoral pulse wave velocity PWV:

$$PWV = \frac{Δ L}{Δ t} = \sqrt{\frac{dP}{ρ} \cdot \frac{V}{dV}} \qquad ( 1 )$$

where
   ΔL: distance separating the two measurement points,
   Δt: timeshift between two waves,
   dP: derivative of the arterial blood pressure with respect to time,
   ρ: blood density,
   V: initial arterial volume,
   dV: derivative of the arterial volume with respect to time.

Therefore the pulse wave velocity (PWV) is a stiffness index. The PWV measurement is inherently non-invasive. It is also a reproducible and perfectly validated technique.

However, its clinical application has been retarded due to the difficulty in obtaining sufficiently precise arterial plots to adequately determine the wave foot. This can be done by mechanograms or by Doppler plots. This work used to be long and tedious until recently. However, recent technological developments enable automatic analysis of plots. Further information on this subject is given in the article “Assessment of arterial distensibility by automatic pulse wave velocity measurement. Validation and clinical application studies” de R. ASMAR et al., Hypertension, 1995, 26, pages 485-490. This should enable more widespread use of PWV in clinical practice.

Contraction of the left ventricle initiates a pressure and deformation wave of the arterial wall that propagates from the heart to the periphery at a finite velocity proportional to the square root of the stiffness of the wall. Many mathematical models have been developed, and can be summarised by the Bramwell and Hill equation (see equation 1). PWV measurements can be based on the transit time of the pressure wave, the flow wave or the deformation wave, all with equivalent results. There are two major points:

(1) The objective is to determine the part of the cardiac cycle in which the PWV measurement is made. It is possible (theoretically or experimentally) to measure the PWV at any point in the cardiac cycle, and therefore at different blood pressure levels. Since arterial distensibility varies as a function of the pressure, any inaccuracy in the pressure will reduce reproducibility.

(2) The objective is to obtain the most precise possible arterial waves particularly in the frequency domain. The PWV is usually determined at the “wave foot”, in other words in diastole. This is the moment in the cardiac cycle at which waves have the most high frequency components. Any damping of the collected waves results in a lack of precision in determination of the wave foot.

At the present time, mechano-transducers with piezoelectric quartz, of the same type as those used for applanation tonometry, are the ideal sensors (due to their high fidelity). Less expensive mechano-transducers with frequency response characteristics compatible with the objectives (0.1 to 100 Hz passband) are also used routinely.

The ease with which the wave foot is legible depends on the frequency response of the transducer and the signal quality. It is obvious that the use of algorithms cannot entirely compensate for bad quality signals. It is essential that the best possible quality pressure waves should be obtained, regardless of whether work is done manually (using the tangents method) or using computer techniques. The use of automatic pulse wave analysis techniques as implemented in the Complior® apparatus (Colson, Les Lilas, France) is a guarantee of excellent reproducibility in measurements.

The measurement of the length travelled by the pressure wave is the weak link in the non-invasive measurement of the arterial stiffness by PWV, particularly sensitive for the carotid-femoral pulse velocity. It is necessary to estimate the length travelled by the pressure wave between the two measurement sites. This is done routinely by measuring the distance on the skin using a meter rule. This approximation was validated in populations with angiographies that can be compared with X-ray data, giving an excellent correlation between the percutaneous length and the length of the arterial tree. However, it is obvious that this is not an ideal choice.

Some populations can induce mismatches depending on the morphology (women with large breasts, obese persons, thorax deformations, etc.), or a disproportionate elongation of the arterial tree (very old patients, megadolichoarteries, etc.). In this case, it is necessary to know how to put the measurement into context. Measurements can be taken between bone reference marks fixed by a system of height gauges, and this approach is now being validated.

The non-homogeneity of the subjacent arterial tree is a criticism frequently made against the velocity of the carotid-femoral pulse wave. The main interest of the carotid-femoral PWV is that it takes account globally of most compliance arteries. However, this magnitude takes account of several arterial segments with different structures, in which the pulse wave propagates in different directions. The thoracic aorta (pure elastic), the abdominal aorta (muscular-elastic), the primitive iliacs and then the external iliacs, and finally the common femoral (muscular) can be seen in the forward direction. The brachiocephalic arterial trunk, and the right common carotid artery are passed through in the reverse direction. The non-homogeneity of the arterial tree is not really a limit to the extent that it translates a physiological-reality (it is what the left ventricle “sees” during ejection) All correction methods to compensate for paths in opposite direction over a short segment induce additional causes of approximation.

The current consensus is that these errors are very marginal, provided that the measurement method is very standardized.

It is possible to measure pressure pulses by applanation tonometry directly. In brief, this technique is based on the applanation principle used by ophthalmologists to measure the intra-ocular pressure. When the chord of a cylinder (or a sphere) is made plane by a plane pressure sensor, the pressure recorded by the sensor is equal to the transmural pressure. H. Millar and M. O'Rourke have developed a pencil probe fitted with a piezoelectric quartz at its tip.

This technique has been validated against intra-aortic measurements. Excellent agreement was demonstrated between carotid pressure pulses measured by tonometry and aortic pressure pulses. It was recently verified on a test bench that the mechanical characteristics of the applanated cylinder have no influence on the absolute value of the transmural pressure. It is also possible to evaluate the morphological characteristics of the pressure wave and to measure reflection phenomena of the pulsatile wave directly.

The pressure wave is propagated from the heart to the periphery at the velocity of the pulse wave. The pressure wave is then reflected on peripheral reflection sites and returns towards the heart. Considering the pulse wave velocity that is of the order of 10-15 m/second and the distance travelled, the reflected wave can be added to the incident wave more or less early within the cardiac systole.

This is very important because the summation of the incident wave and the reflected wave during systole increases the work done by the heart and reduces the coronary perfusion pressure (the coronary perfusion occurring during diastole). On the contrary, the late return of the reflected wave after closure of the aortic sigmoids increases the coronary perfusion pressure and limits the work done by the heart.

The following factors determine early return of the reflected wave:
the increase in arterial stiffness,
the small size,
significant conisation of the aorta,
connection angles between open collaterals,
peripheral vasoconstriction,
bradycardia.

Several parameters are used to quantify the intensity of the reflection wave. They all make use of the central pressure wave analysis using Murgo's nomenclature. Information about this subject is found in the article entitled “Manipulation of ascending aortic pressure and flow wave reflections with the Valsalva maneuver: relationship to input impedance” by J. P. MURGO et al., Circulation, January 1981, 63(1), pages 122-132.

FIGS. 2 and 3 illustrate Murgo's classification for aortic pressure waves. FIG. 2 represents a type A pressure wave characteristic of an elderly patient with hypertension. FIG. 3 represents a type C pressure wave, characteristic of a young patient in good physical condition.

The ratio between ΔP and PP is called the augmentation index. Δtp is the time to the shoulder. The ventricle ejection time is called LVET (“left ventricle ejection time”). Pi is the pressure at the inflection point.

The amplitude of the pressure pulse and the augmentation index are direct estimates of the intensity of the reflection wave. The time to the shoulder evaluates the distance of the reflection sites. Finally, the ventricle ejection time provides useful information in itself.

We have seen that the determinants of each of these parameters are varied and that the arterial stiffness is only one of the parameters involved. Therefore it is inaccurate to claim that the augmentation index is a pure arterial stiffness parameter (regardless of how useful it is for other purposes).

All techniques that can be used for this type of study are based on applanation toponometry. The difference depends on the pressure signal collection site and signal analysis techniques. Ideally, the pressure wave must be collected as close as possible to the aortic valves. The carotid pressure wave is a good compromise using a non-invasive method. Pressure plots may be analysed manually (on plots) or digitised. The advantage of the augmentation index is that it is non-dimensional (eliminating all calibration problems).

FIG. 4 shows a diagrammatic view of the incident wave 1 and the reflected wave 2. The summation of the two wave trains determines the morphology of the observed pressure wave. For subjects with very distensible arteries, the summation is made in diastole (case of curve 3). If the arteries are rigid, the summation is made during systole and increases the pressure accordingly (see curve 4).

It is theoretically possible to start from a peripheral pressure plot (like the radial artery) using transfer function techniques to reconstitute the carotid or even the aortic pressure wave. Such a transfer function was created within a normal reference population. It works reasonably well for comparable populations, but there is no way of being certain that this transfer function can be extrapolated to ill populations. An apparatus has been marketed based on this principle, with the tradename Sphygmocor® (PWV Medical, Sydney, Australia).

Only one apparatus has been marketed to measure the pulse wave velocity. This is the Complior® apparatus marketed by the ARTECH-MEDICAL company. Distribution of these techniques is limited by several characteristics of this apparatus. First it is expensive. It uses large volume membrane mechano-sensors that are not very sensitive, not very accurate and cannot be used for a detailed analysis of the pressure wave. It is a special-purpose apparatus; it can only be used to measure the pulse wave velocity and no other arterial parameter derived from the analysis of the pressure wave. Finally, it is complicated to learn because it is difficult to handle its sensors.

Applanation tonometry was initially described for ophthalmological applications (measurement of the intra-ocular pressure). It was adapted to non-invasive arterial hemodynamics in the 1980s. It is possible to use these techniques to non-invasively collect the pressure wave, calculate amplification indexes and measure the pulsatile pressure at every palpable surface artery. In short, when the arc of a segment of a cylinder is made plane by a pressure sensor, the transmural pressure recorded by the said sensor is equal to the intravascular pressure. The Millar company has developed a high fidelity piezoelectric quartz sensor mounted on a pencil probe for making this type of measurement. This type of apparatus is very expensive, particularly because of the need for an acquisition control unit and possibly the need to acquire algorithms for calculation of the central pressure pulse and amplification indexes as implemented in the Sphygmocor® system. Furthermore, the nature of the sensor mounted on a pencil makes it impossible to take a fine palpation of the pulse at the same time as the sensor is being positioned, so that this technique is very dependent on the operator.

At the present time, only specialised research centres are capable of measuring the pulse wave velocity and the carotid pressure curve, or using these values to predict risks.

Thus, there are three types of limits for the diffusion of techniques:

technological: large mechanical sensors, difficult to handle, are inserted between the signal to be collected (arterial pulse) and the touch sensitivity of the operator;

methodological: difficult methodological learning, not easily compatible with normal medical exercise, measurement quality highly dependent on the operator, raw results without context and difficult to interpret;

economic: existing apparatuses are prototypes or small series specially used for clinical research, perform a single function and are expensive.

### PRESENTATION OF THE INVENTION

This invention was designed to overcome the disadvantages of prior art.

One purpose of the invention consists of a blood pressure microsensor comprising means of holding and positioning a chip of piezoresistive material on the inside face of a practitioner's finger, the chip dimensions being smaller than the diameter of the artery in which the pressure is to be measured, the microsensor also comprising means of transmitting the electrical signal output by the piezoresistive chip in response to a pressure applied to the chip.

Advantageously, the means of holding and positioning the chip of piezoresistive material are composed of a glove finger onto which the chip is fixed.

A second purpose of the invention consists of an apparatus for measuring the arterial stiffness comprising:
a first arterial pressure microsensor like that defined above, used to make a blood pressure measurement at a first determined location on a patient's body,
a second arterial pressure microsensor like that defined above, used to make a blood pressure measurement at a second determined location on a patient's body different from the first determined location,
a processing and calculation device receiving input consisting of electrical signals output by the first and the second pressure microsensor and information about the arterial circulation length between the first determined location and the second determined location, the device being provided with calculation means for calculating the patient's pulse wave velocity and using it to deduce the patient's arterial stiffness, making use of the electrical signals and the information received as input.

Advantageously, the determined locations correspond to the primitive carotid artery at the neck and the common femoral artery at the groin.

The device may also be provided with evaluation means for providing an indication of risks of cardiovascular accidents of a patient as a function of the deduced arterial stiffness, and other risk factors.

### BRIEF DESCRIPTION OF THE DRAWINGS

The invention will be better understood and other advantages will become clear after reading the following description given as a non-limitative example, accompanied by the attached drawings among which:

FIG. 1, already described, illustrates the technique used to measure the carotid-femoral pulse wave velocity,

FIGS. 2 and 3, already described, illustrate the Murgo classification for aortic pressure waves,

FIG. 4, already described, is a diagrammatic representation of an incident wave and a reflected wave, for the blood pressure,

FIG. 5 shows a chip of piezoresistive material held and positioned on a glove finger and forming part of the arterial pressure microsensor according to the invention,

FIG. 6 shows an arterial stiffness measurement apparatus according to the invention.

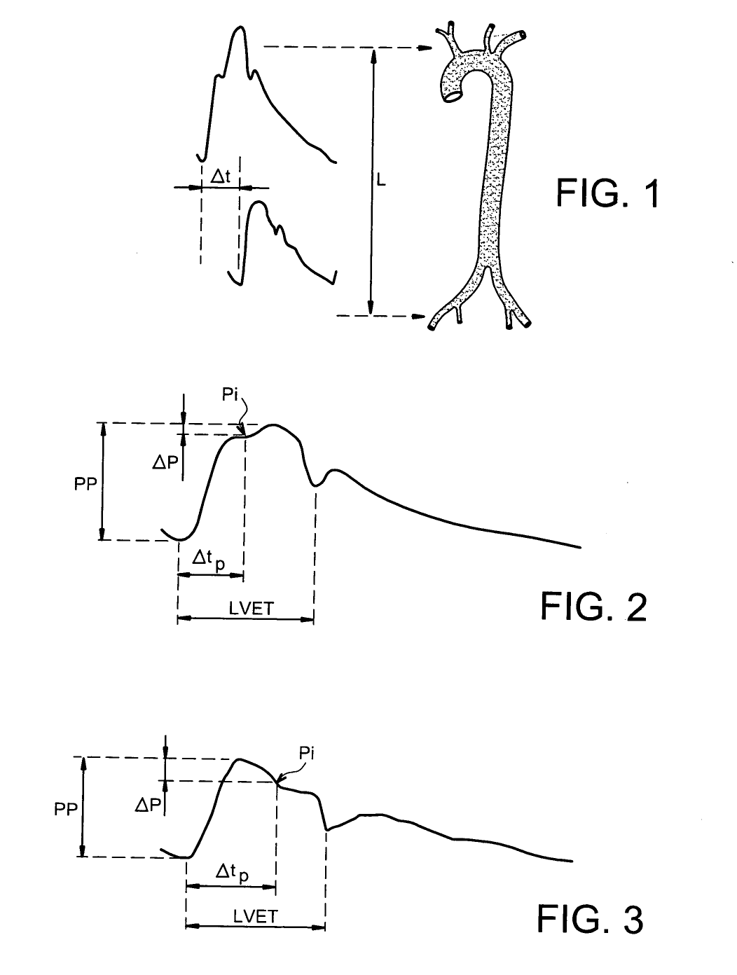

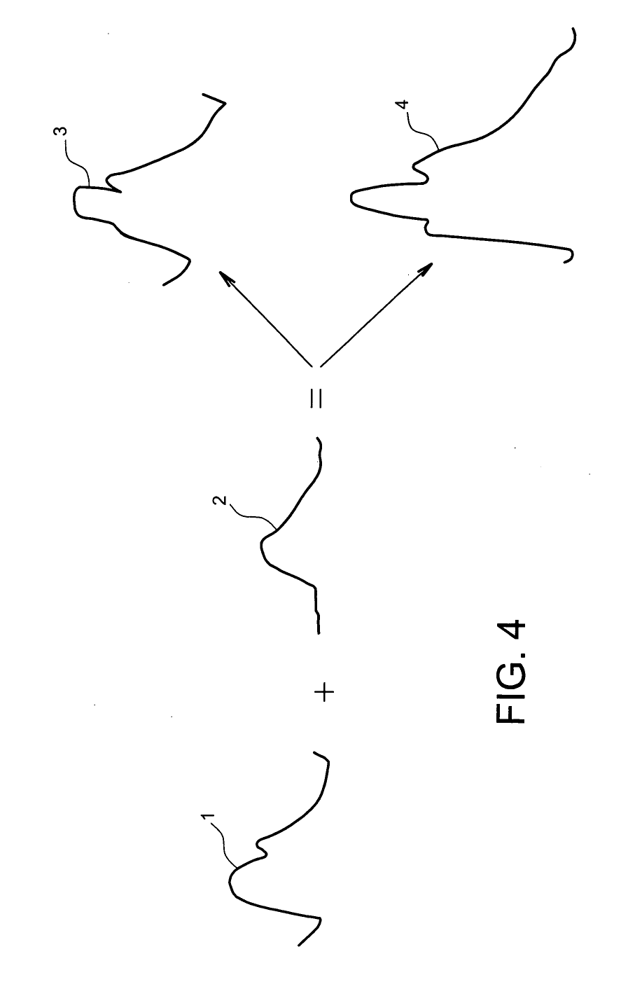

### DETAILED DESCRIPTION OF EMBODIMENTS OF THE INVENTION

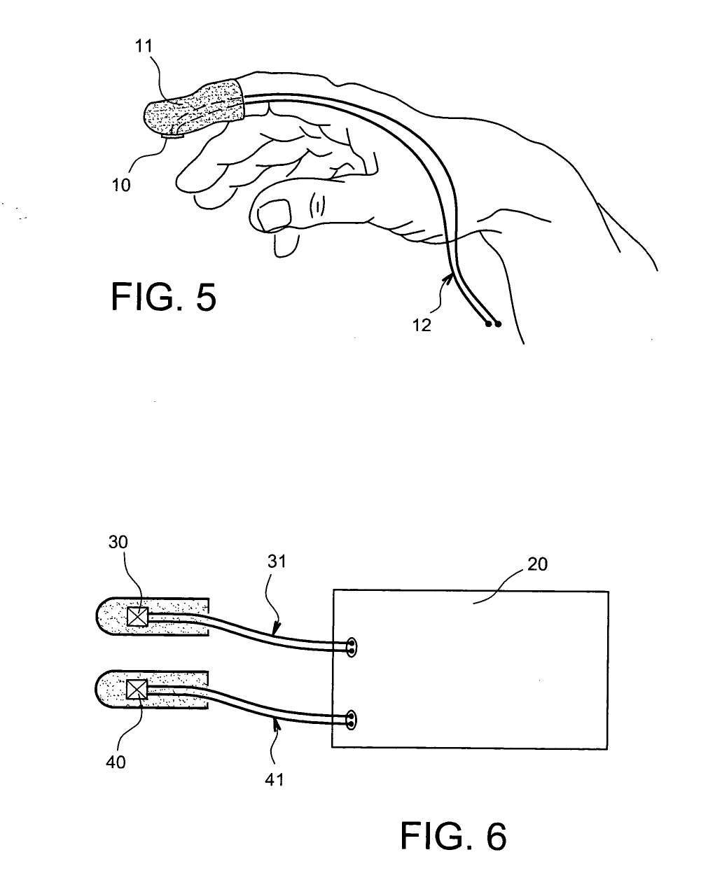

FIG. 5 shows a chip of piezoresistive material 10, held and positioned on a glove finger 11 located on a practitioner's finger. Electrical conductors 12 electrically connect the chip of piezoresistive material to a system for interpretation of electrical signals transmitted by the chip. The dimensions of the chip 10 are smaller than the diameter of the artery for which the pressure is to be measured.

For example, the material from which the chip is made may be of the piezo-resistive type. With the chip, a very small measurement area (about 2 mm) can be achieved so that the point to be measured can be very precisely positioned. Any important difference between the measurement point and the point to be measured will result in severe attenuation of the collected signal.

The microsensor according to the invention simultaneously controls measurement of the electronics and fine palpation of the pulse by the practitioner. It can be used to measure the pressing surface pulse and the deep-seated pulse while the pressing force is controlled manually.

The microsensor according to the invention induces is a very small deformation in the artery to be measured, unlike sensors used at the present time. Therefore there are no major disturbances to the fluid mechanics in the artery to be measured.

The microsensor according to the invention can be used to make pulse measurements at places on the body that are difficult to measure using sensors according to prior art.

The sensor chip can be inserted inside a glove finger to facilitate the measurement. The chip may be on the inside or the outside of the glove finger. It may be put into place by deposition.

The chip may also be insert moulded to obtain a hard part facilitating good localisation.

FIG. 6 shows an apparatus for measuring the arterial stiffness according to the invention comprising a processing and calculation device 20, receiving input consisting of electrical signals output by first and second blood pressure microsensors. The first microsensor comprises a first chip 30 made of a piezoresistive material 30 connected to the apparatus 20 by electrical conductors 31. The second microsensor comprises a second chip 40 made of a piezoresistive material connected to the apparatus 20 by electrical conductors 41. For example, the first microsensor is designed to measure the pressure of the primitive carotid artery at the neck. The second microsensor is intended for example to measure the pressure of the common femoral artery at the groin.

The processing and calculation device 20 also receives information about the blood circulation length between two pressure measurement points. It has calculation means for calculating the pulse wave velocity of a patient starting from data entered through its inputs. It then outputs a value of the patient's arterial stiffness.

Electrical signals transmitted by the microsensors may be formatted for interpretation by a digital acquisition system connected to a computer system, at the input to device 20. This computer system may be small or it may be coupled to a portable computer or to any other signal processing or transmission device.

The pulse wave velocity measurement may be coupled with the carotid pulse wave analysis.

The measurement of the distance between the two measurement points may be considerably facilitated and improved by using an attached ultrasound sensor.

It has been demonstrated that the predicted value provided by a measurement of the arterial stiffness using the pulse wave velocity is equal to or greater than the values available using the Framingham algorithm. It has also been demonstrated that the value predicted by the combined use of the pulse wave velocity and the Framingham algorithm is better than is possible using either of the parameters taken separately. The proposed score is derived from statistical logical regression models taking account of these two measurements. The values of each coefficient used in this logistical regression model are derived from an epidemiological study. Information about this subject is given in the article by P. BOUTOUYRIE et al. entitled [symbol] Aortic stiffness is an independent predictor of primary coronary events in hypertensive patients: a longitudinal study [symbol] published in Hypertension, January 2002, 39 (1): 10-5.

The sensor and its measurement device may be useful particularly when there is a need for a remote diagnostic for an isolated patient, and particularly for an offshore race or an expedition, a space voyage, on a drilling platform, for speleology or to handle emergency or routine situations.

1. Blood pressure microsensor comprising means of holding and positioning a chip of piezoresistive material (10) on the inside face of a practitioner's finger, the chip dimensions being smaller than the diameter of the artery in which the pressure is to be measured, the microsensor also comprising means of transmitting the electrical signal output (12) by the piezoresistive chip in response to a pressure applied to the chip (10).

2. Microsensor according to claim 1, characterised in that the means of holding and positioning the chip of piezoresistive material (10) are composed of a glove finger (11) onto which the chip is fixed.

3. Apparatus for measuring the arterial stiffness comprising:
a first arterial pressure microsensor (30, 31) according to either claims 1 or 2, for a blood pressure measurement at a first determined location on a patient's body,
a second arterial pressure microsensor (40, 41) according to either claims 1 or 2, for a blood pressure measurement at a second determined location on a patient's body different from the first determined location,
a processing and calculation device (20) receiving input consisting of electrical signals output by the first and the second pressure microsensor and information about the arterial circulation length between the first determined location and the second determined location, the device being provided with calculation means for calculating the patient's pulse wave velocity and using it to deduce the patient's arterial stiffness, making use of the electrical signals and the information received as input.

4. Device according to claim 3, characterised in that it is also provided with evaluation means for providing an indication of risks of cardiovascular accidents of a patient as a function of the deduced arterial stiffness.
